(19) United States
(12) Patent Application Publication
(10) Pub. No.: US 2012/0215300 A1
(43) Pub. Date: Aug. 23, 2012

(54) METHOD AND APPARATUS FOR BYPASS GRAFT

(76) Inventors: Charles L. Richardson, Monroe, NC (US); Franz W. Kellar, Gastonia, NC (US); Donald G. Faulkner, Charlotte, NC (US)

(21) Appl. No.: 13/463,604

(22) Filed: May 3, 2012

Related U.S. Application Data
(63) Continuation of application No. 11/857,317, filed on Sep. 18, 2007, now abandoned.

(51) Int. Cl. A61F 2/06; B23K 9/02

(52) U.S. Cl. 623/1.36; 219/60.2

(57) ABSTRACT

A vascular connector includes a main tube having a channel for fluid flow therethrough and opposed ends adapted to be connected to a vascular structure; and at least one inlet tube having a channel for fluid flow therethrough, a proximal end intersecting the main tube, and a distal end adapted to be connected to a vascular structure.

### CROSS-REFERENCE TO RELATED APPLICATIONS

This application is a Continuation of application Ser. No. 11/857,317, which is currently pending.

### BACKGROUND OF THE INVENTION

This invention relates generally to bypass grafts and more particularly to devices and methods for coronary bypass grafts.

Coronary artery disease is a major medical problem, resulting in frequent hospitalization and death. It occurs when there is a narrowing in one of the heart's arterial systems that supplies oxygenated blood to the heart muscle. The resulting loss of blood flow causes a loss in heart capacity. If an artery becomes completely blocked a heart attack will result.

It is known to surgically treat coronary artery disease using coronary artery bypass grafts (“CABG”). In this procedure, vessels harvested from another part of the patient's body are used to construct a bypass route from the aorta to a point downstream of the narrowing or blockage.

Existing grafts are difficult to implement, requiring careful measurement, and traumatic harvesting of vessels from the patient. Furthermore, known techniques of connecting blood vessels to each other do not result in hydrodynamically ideal flow configurations of the connected vessels. This can cause turbulence and restricted flow in the bypass graft.

### BRIEF SUMMARY OF THE INVENTION

These and other shortcomings of the prior art are addressed by the present invention, which according to one aspect provides a vascular connector, having: a main tube having a channel for fluid flow therethrough and opposed ends adapted to be connected to a vascular structure; and at least one inlet tube having a channel for fluid flow therethrough, a proximal end intersecting the main tube, and a distal end adapted to be connected to a vascular structure, wherein the geometry of the intersection between the at least one inlet tube and the main tube is configured to enhance mixing of fluid flowing from the inlet tube with fluid flowing in the main tube.

According to another aspect of the invention, a method is provided for manufacturing a vascular connector which includes a main tube having a channel for fluid flow therethrough and opposed ends adapted to be connected to a vascular structure, and at least one inlet tube having a channel for fluid flow therethrough, a proximal end intersecting the main tube, and a distal end adapted to be connected to a vascular structure. The method includes: providing a generally planar blank; forming the blank into a shape comprising mirror-image half-sections of the main tube and inlet tube; folding the blank along a centerline thereof to bring the half-sections together; and bonding the free edges of the folded blank together.

According to another aspect of the invention, a coronary artery bypass graft includes: a substantially rigid connector having: a main tube with first and second ends; and an inlet tube having a proximal end intersecting the main tube, and a distal end adapted to be connected to a vascular structure; synthetic vessel having a proximal end adapted to be connected to a first vascular structure, and at least one distal end connected to and the distal end of the inlet tube.

According to another aspect of the invention, a connection for a vascular structure includes: a generally tubular fitting having a first end adapted to be secured to a vascular structure and a second end having a mechanical fitting; and a synthetic vessel adapted to be connected to the mechanical fitting.

According to another aspect of the invention, a method of monitoring a vascular graft structure includes: providing a substantially rigid connector having: a main tube with first and second ends; and at least one inlet tube having a proximal end intersecting the main tube, and a distal end; connecting the first and second ends of the main tube to a first vascular structure; connecting the distal end of the at least one inlet tube to a distal end of a graft vessel; connecting a proximate end of the graft vessel to a second vascular structure; providing at least one transducer operable to sense a parameter related to flow to one of the graft vessel or the connector; and monitoring a parameter related to flow through the vascular graft structure to determine whether the vascular graft structure is performing properly.

According to another aspect of the invention, a cutting device for a vascular structure includes: an open-ended chamber which supports a shaft for rotation and axial translation therein; a blade having a circular cutting edge carried at a lower end of the shaft; and a port passing through a wall of the chamber for connection of a suction source.

### BRIEF DESCRIPTION OF THE DRAWINGS

The invention may be best understood by reference to the following description taken in conjunction with the accompanying drawing figures in which:

FIG. 1 is a front view of a heart having a coronary artery bypass graft constructed according to an aspect of the present invention connected thereto;

FIG. 2 is a perspective view of a vascular connector for the bypass graft;

FIG. 3A is a cross-sectional view of the connector of FIG. 2;

FIG. 3B is a side view of the connector of FIG. 2;

FIG. 4 is an enlarged view of a portion of the connector of FIG. 3;

FIG. 5 is a perspective view of an alternative connector;

FIG. 6 is an end view of the connector of FIG. 5;

FIG. 7 is cross-sectional view taken along lines 7-7 of FIG. 6;

FIG. 8 is an end view of another alternative connector;

FIG. 9 is cross-sectional view taken along lines 8-8 of FIG. 7;

FIG. 10A is a perspective view of another alternative connector having a helical inlet;

FIG. 10B is a cross-sectional view of a portion of the connector of FIG. 10A;

FIG. 11 is a perspective view of another alternative connector having a wire mesh vessel connection;

FIG. 12 is a schematic cross-sectional view of a connector having one leg blocked;

FIG. 13 is a schematic cross-sectional view of a connector having one leg blocked with a bleed orifice therein;

FIGS. 14A and 14B are top and end views, respectively, of a blank for a connector in a first step of manufacture;

FIGS. 15A and 15B are top and end views, respectively, of a blank for a connector in a subsequent step of manufacture;

FIGS. 16A and 1B are side and end views, respectively, of a blank for a connector in a final step of manufacture;

FIG. 17 is a perspective view of a synthetic vessel for use with the present invention;

FIG. 18 is a perspective view of a heart having the vessel of FIG. 17 connected thereto;

FIG. 19 is a schematic cross-sectional view of an aortic connection constructed in accordance with an aspect of the present invention;

FIG. 20A is a schematic cross-sectional view of an alternative aortic connection;

FIG. 20B is a top view of a connector flange of the connector of FIG. 20A;

FIG. 21 is a schematic cross-sectional view of a cutting tool for use with the aortic connections shown in FIGS. 19 and 20; and

FIG. 22 is a side view of a vascular connector with a transducer attached thereto.

### DETAILED DESCRIPTION OF THE INVENTION

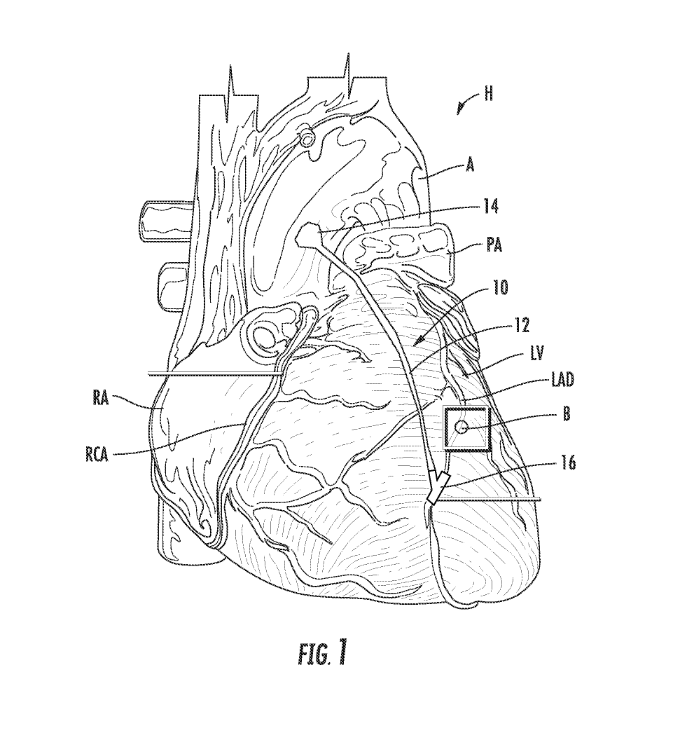

Referring to the drawings wherein identical reference numerals denote the same elements throughout the various views, FIG. 1 shows a heart “H” including the left ventricle “LV”, right atrium “RA”, left pulmonary artery “PA” and aorta “A”. The left anterior descending artery “LAD” and right coronary artery “RCA” extend down the front surface of the heart H. Each of these arterial structures has multiple branches which supply oxygenated blood to the heart muscle tissue. Frequently the LAD or RCA will become partially or totally occluded, preventing normal operation, for example by a blockage at point “B”. A coronary artery bypass graft (CABG) 10 according to the present invention is implemented on the illustrated heart H. the CABG includes a graft vessel 12 which extends between an aortic connection 14 and a connector 16. The connector 16 provides a fluid connection between the graft 14 and a vessel (i.e. a portion of the LAD or RCA) downstream of the blockage B. While the present invention is described in the context of a coronary graft, the techniques and devices described herein may also be used any other kind of fluid bypass structure within a human or animal body.

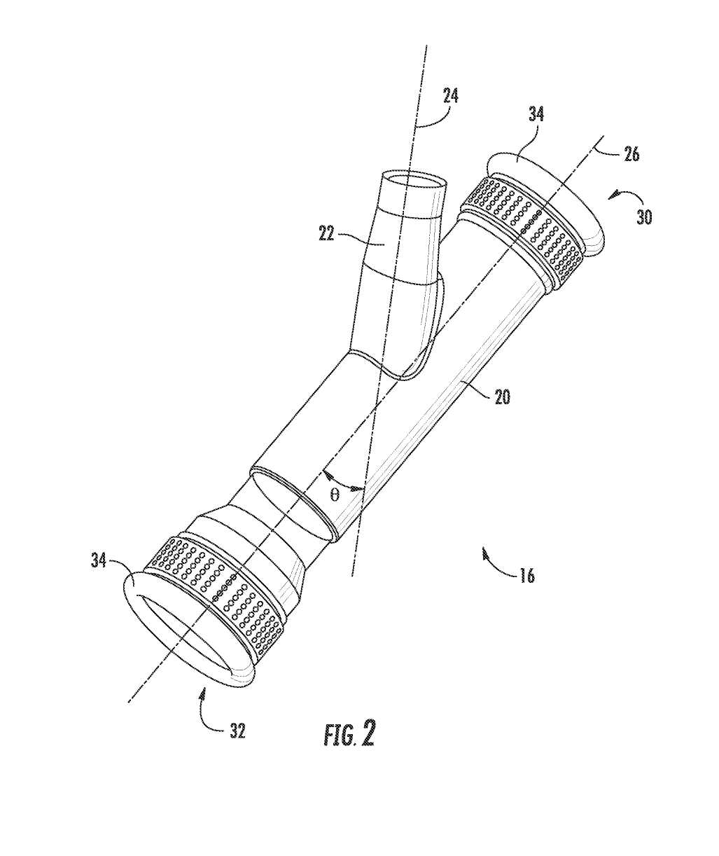

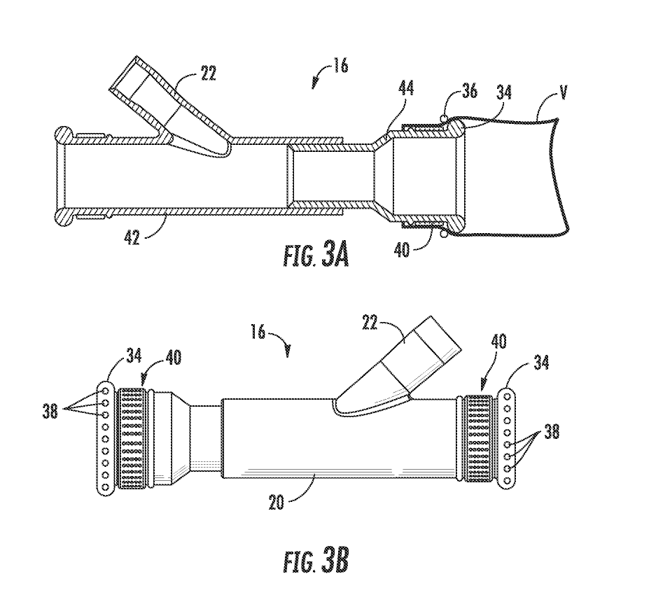

The connector 16 is shown in FIGS. 2, 3A, and 3B. It is generally tubular in construction and includes a main tube 20 and at least one inlet tube 22. The tubes 20 and 22 may have circular, elliptical, or varying cross-sections as described in more detail below. The central axis 24 of the inlet tube 22 is disposed at an angle θ to the central axis 26 of the main tube 20, to enhance mixing of fluid from the inlet tube 22 to the main tube 20 and to accommodate the physical attachment of the graft (vessel) to the inlet tube 22. Suitable values for angle θ may be from about 0° to about 90°. While angle θ may be varied to suit a particular application, lower values of 0 generally provide better flow mixing. In the illustrated example, angle θ is about 30°

The main tube 20 has first and second ends 30 and 32 which are adapted to create a leak- and strain-free surgical connection to a blood vessel. As illustrated, each end 30 and 32 includes an outer rim 34 of increased diameter. A suture ring 36, elastic band, other type of closure or surgical adhesive is used to cinch a vessel, shown at “V” in FIG. 3A, around the outer rim 34. The outer rim 34 may also include a series of holes 38 that sutures can be passed through. For a more permanent connection, each end of the connector 16 also includes a “strain relief” zone 40 that may be covered with a collagen-hydroxyl-apatite tape supporting a suitable fibrous scaffolding for the promotion of tissue growth and stabilization from the existing tissue of the vessel V. The fiber scaffolding could also be “seeded” with human stem cells or other suitable materials to promote tissue growth and long term stabilization if required. Other materials such as fiber flock, wire mesh, or GORE-TEX microporous PTFE fabric may also be used in the strain relief zones 40 to provide sites for tissue growth.

If additional strain relief or attachment security is required for the connector 16, then it may also be covered with a thin perforated shaped disk (not shown) placed over the connector 16 using the exposed leg of the inlet tube 22 for location and positional registration. This disk would be sutured in situ. The underside of the shaped disk would be covered with a collagen-hydroxyl-apatite tape supporting a suitable fibrous scaffolding for the promotion of tissue growth and stabilization from the existing surrounding tissue. It is also envisioned that the fiber scaffolding could also be “seeded” with human stem cells or other suitable materials to promote tissue growth and long term stabilization if required.

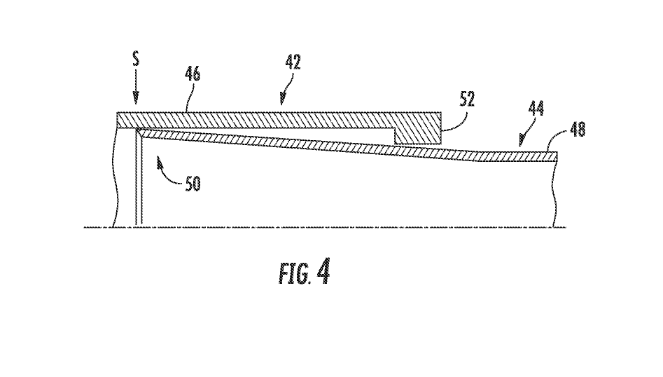

The main tube 20 may be built up from first and second members 42 and 44 which fit together in a telescoping friction fit. This arrangement allows the overall length of the main tube 20 to be varied, and also permits relative rotation of its first and second ends 30 and 32. This greatly eases attachment of the connector 16 to vessels V in a stress-free fit, because the length of the gap to be spanned and the relative angular orientations of the cut ends of the vessel V are not critical. FIG. 4 shows this telescoping fit in more detail. The wall 46 of the first member 42 is generally of a constant inside diameter. The wall 48 of the second member 44 is tapered, with its greatest outer diameter at its distal end 50. This diameter is selected to be a close sliding fit or light interference with the inside diameter of the first member 42. When assembled, this approximates an annular line contact which seals tightly against leakage (see arrow “S”) while still permitting sliding and rotation of the first and second members 42 and 44. If desired, the first member 42 may include a flange 52 to prevent complete withdrawal of the second member 44 in use. The direction of overlap of the first and second members 42 and 44 may be reversed, i.e. the second member 44 may have the larger diameter of the two mating components. Furthermore, the inlet tube 22 may incorporate a similar telescoping structure if desired.

The connector 16 may be constructed from any material which is biologically inert or biocompatible and will maintain the desired shape when implanted. Examples include metals and biocompatible plastics. One example of a suitable material is an alloy of nickel and titanium generally referred to as NITINOL. Other known metals used for implants include titanium, stainless steels, cobalt chrome, cobalt-chromium-molybdenum, titanium-aluminum-niobium and similar materials.

The connector 16 is shaped and sized to efficiently mix the flow from the inlet tube 22 into the main tube flow by providing low stagnation flow, low to zero turbulence, laminar flow, and low impingement flow. One specific way this is implemented is by shaping of the junction of the inlet tube 22 and the main tube 20. As shown in FIGS. 2 and 3, the portion of the inlet tube adjacent to the main tube 20 is flattened into an elliptical shape to direct inlet flow in a relatively narrow jet adjacent the inner wall of the main tube 20. This helps to avoid turbulent mixing. If needed, a shaped metering orifice (e.g. converging-diverging) may be incorporated into the inlet tube. This slightly dampens upstream pressure or reduces the blood flow level to limit vascular stress and flow turbulence at the intersection of the inlet and main tubes 22 and 20, the transitions between telescoping sections of the connector 16 and potentially the transition between the ends of the connector 16 and the attached vascular structure. Depending on the particular application, the geometry of the inlet tube 22 and main tube 20 could be configured for laminar flow, turbulent flow, or mixed flow.

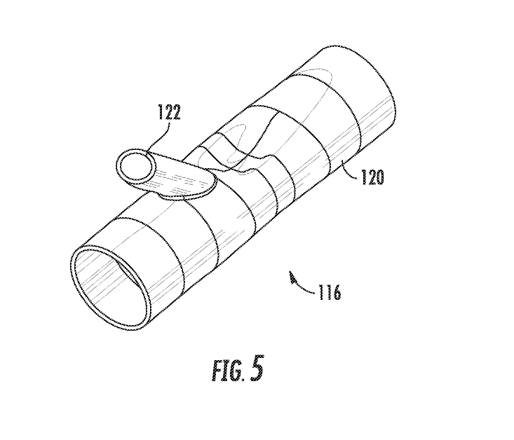

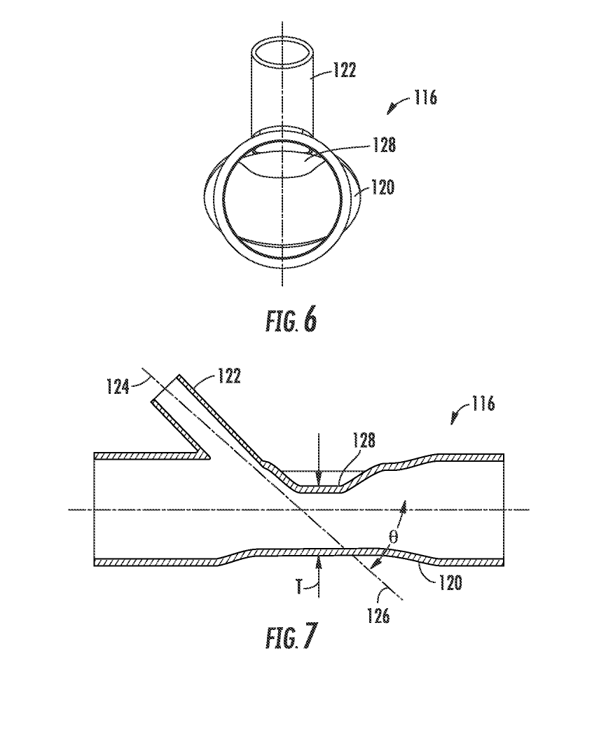

FIGS. 5, 6, and 7 illustrate an alternative connector 116. It is generally similar in construction to the connector 16 and includes a main tube 120 and at least one inlet tube 122. The tubes 120 and 122 may have circular, elliptical, or varying cross-sections as described in more detail below. The central axis 124 of the inlet tube 122 is disposed at an angle θ to the central axis 126 of the main tube 120, to enhance mixing of fluid from the inlet tube 122 to the main tube 120 and to accommodate the physical attachment of the graft vessel to the inlet tube 122. Suitable values for angle θ may be from about 0° to about 90°. While angle θ may be varied to suit a particular application, lower values of 0 generally provide better flow mixing. In the illustrated example, angle θ is about 30°. The connector 116 differs from the connector 16 in that the main tube 120 incorporates a bulge or protrusion 128 which defines a minimal cross-sectional area or throat “T” downstream of the discharge plane of the inlet tube 122. This area reduction causes a velocity increase and attendant pressure drop which tends to draw fluid into the main tube 120, from the inlet tube 122, improving mixing of the two fluid streams while discouraging turbulence.

While not shown in the Figures, the connector 116 may incorporate the attachment structures and the telescoping configuration described above for the connector 16.

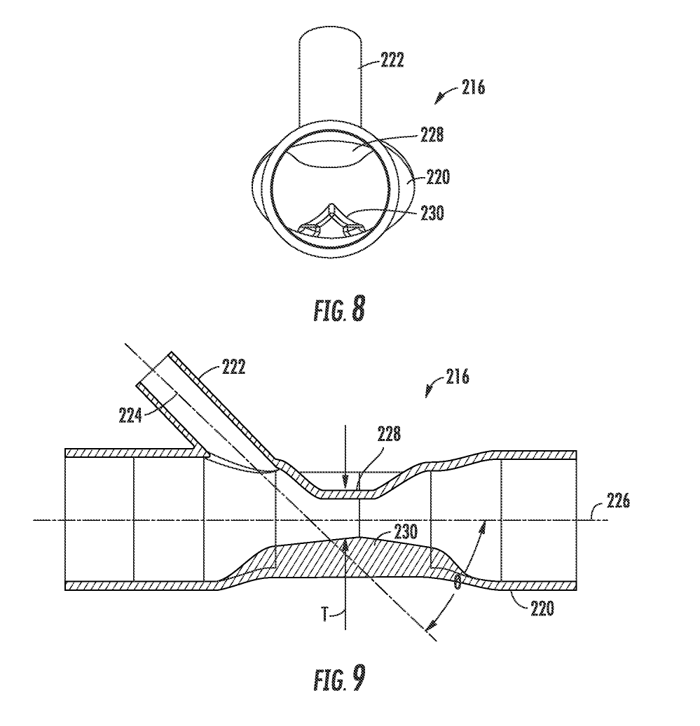

FIGS. 8 and 9 illustrate another alternative connector 216 which is generally similar in construction to the connector 116. It includes a main tube 220 and at least one inlet tube 222. The tubes 220 and 222 may have circular, elliptical, or varying cross-sections. The central axis 224 of the inlet tube 222 is disposed at an angle θ to the central axis 226 of the main tube 220, to enhance mixing of fluid from the inlet tube 222 to the main tube 210 and to accommodate the physical attachment of the graft (vessel) to the inlet tube 222. Suitable values for angle θ may be from about 0° to about 90°. While angle θ may be varied to suit a particular application, lower values of 0 generally provide better flow mixing. In the illustrate example, angle θ is about 30°. The connector 216 incorporates a bulge or protrusion 228 which defines a minimal cross-sectional area or throat “T” downstream of the discharge plane of the inlet tube 122, as with the connector 216. In addition, the connector 216 includes a flow splitter 230 disposed on the wall of the main tube 220 opposite the protrusion 228. As best seen in FIG. 8, the flow splitter 230 has opposed concave faces. In combination with the area reduction, this shaping tends to set up a pair of opposed laminar vortices in the flow in the main tube 220; this in turn draws in flow from the inlet tube 222 while maintaining stream integrity and flow efficiency with minimal disruptions. The connector 216 may also incorporate the attachment structures and the telescoping configuration described above for the connector 16

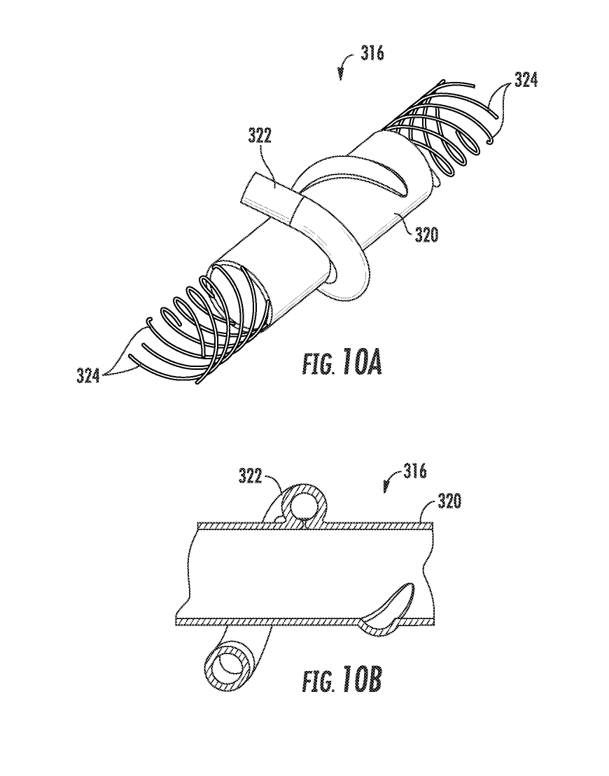

FIGS. 10A and 10B illustrate another alternative connector 316. It includes a main tube 320 and at least one inlet tube 322. The tubes 320 and 322 may have circular, elliptical, or varying cross-sections. The inlet tube 322 wraps around the main tube 320 has a spiral or helical shape of variable pitch which gradually transitions flow from a tangential direction to an axial direction as it mixes with the flow in the main tube 320. The connector 316 may also incorporate the attachment structures and the telescoping configuration described above for the connector 16. In the particular example illustrated, the connector 316 has a series of spiral wires 324 protruding from each end to serve as a blood vessel attachment scaffolding.

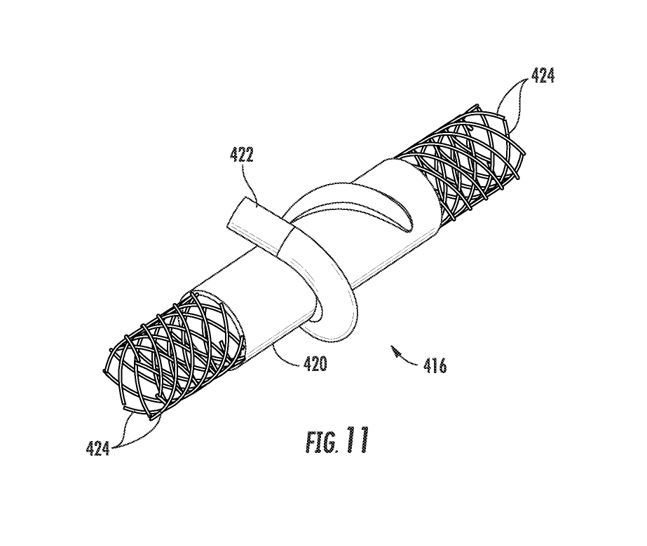

FIG. 11 illustrates yet another alternative connector 416. It is substantially identical in construction to the connector 316 and includes a main tube 420 and at least one inlet tube 422. It differs in that it includes two nested series of spiral wires 424 protruding from each end. These collectively form a wire mesh which serves as a blood vessel attachment scaffolding.

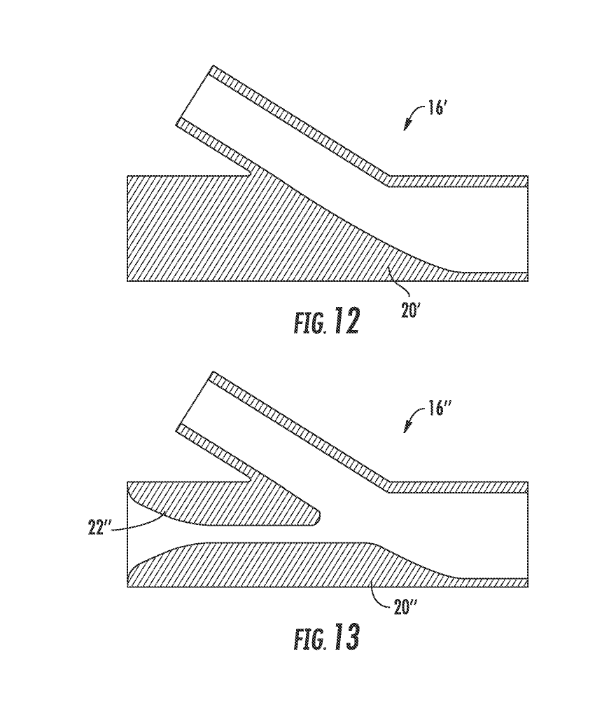

The connectors described above are illustrated with their respective main tubes completely open to flow. However, depending upon the condition of the particular patient, it may be desirable to block of flow from the vessel that is being bypassed. FIG. 12 illustrates a generic connector 16′ in which the upstream end of the main tube 20′ is blocked off. This feature may be implemented with any of the connectors described above. Alternatively, it may be desirable to substantially block flow from the bypassed vessel while allowing some flow to prevent total flow stagnation and pooling of fluid. FIG. 13 illustrates another generic connector 16″ which has the upstream end of the main tube 20″ blocked off except for a calibrated orifice 22″ which permits a metered amount of flow from the bypassed vessel.

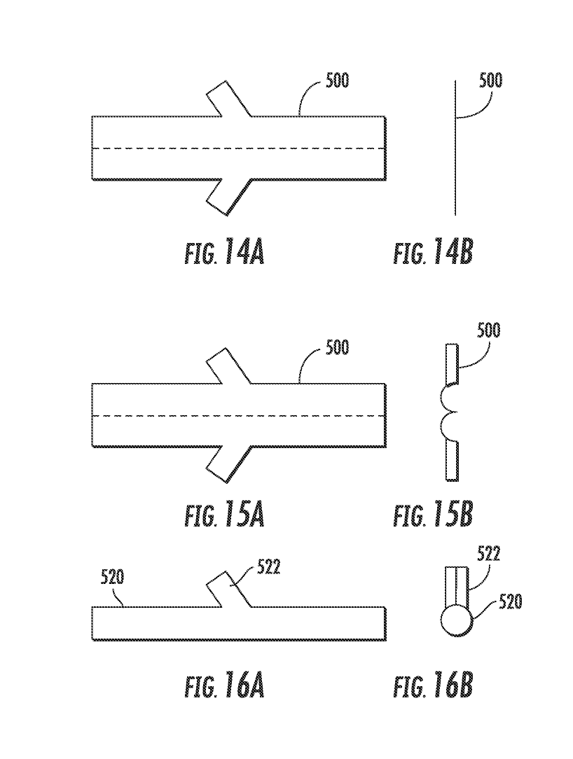

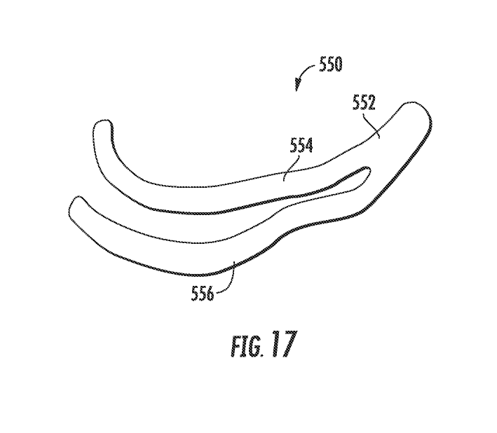

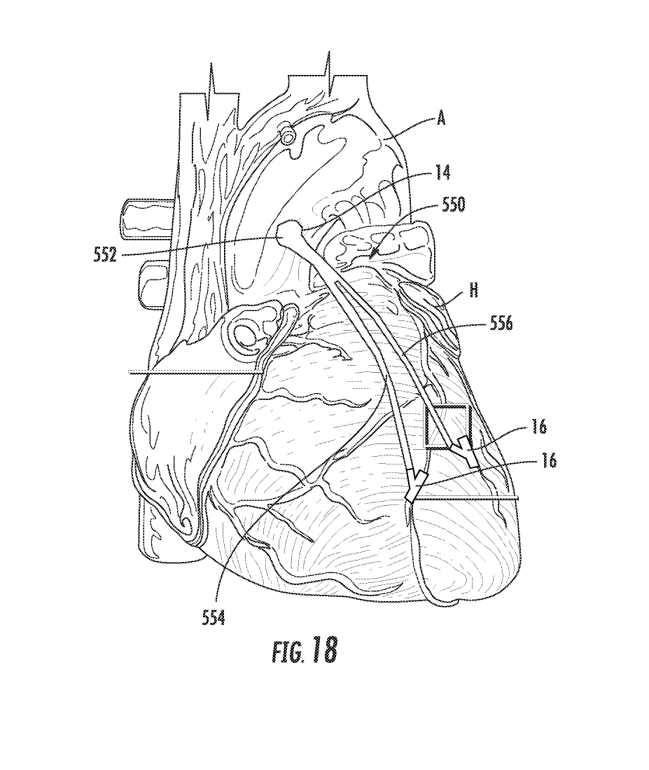

The connectors described above may be manufactured using a variety of techniques, for example by machining, extruding, or injection molding. FIGS. 14 through 16 illustrate sequential steps in a method that is believed to be especially useful. First, as shown in FIGS. 14A and 14B, a flat blank 500 with mirror-image halves is stamped or cut from sheet-like material. Optionally, the blank 500 may then be coated with a biocompatible or biologically inert coating. Next, the blank 500 is formed, for example using stamping dies, to form symmetrical half-sections of the desired shape, as shown in FIGS. 15A and 15B. Next, the blank 500 is folded in half to form a connector with a main tube 520 and an inlet tube 522 (FIGS. 16A and 16B). The open (free) edges of the main and inlet tubes 520 and 522 are bonded together, for example with an adhesive, crimping, thermal bonding, electron-beam welding, or the like. Optionally, the interior of the connector may be finished in a known process in which a viscous abrasive media is flowed through its interior passages.

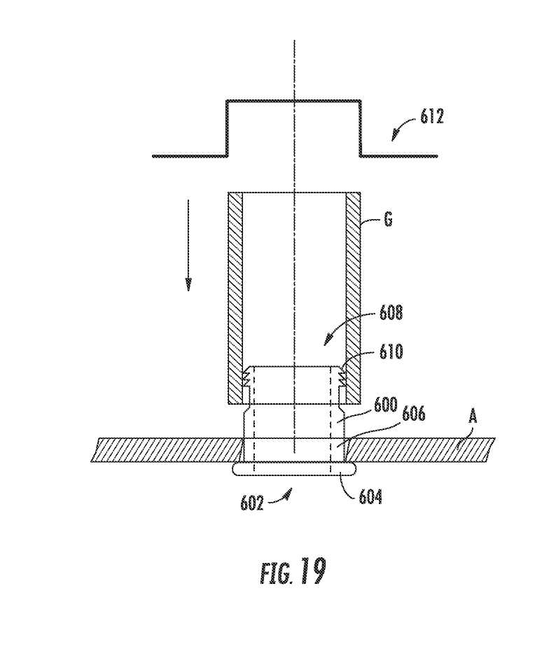

The connectors described above may be used with natural vessel or synthetic vessel grafts. FIG. 17 illustrates a synthetic vessel 550 having a trunk 552 and two or more branches 554 and 556. If more than one bypass is required, it can be accomplished using with only a single aortic connecting by using the vessel 550. For example, FIG. 18 illustrates a CABG on a heart H. The vessel 550 is joined to the aorta A at an aortic connection 14. One of its branches 554 is connected to one leg of the LAD via a first connector 16 and another branch 556 is connected to another leg of the LAD via a second connector 16.

FIG. 19 illustrates one method of making an aortic connection. An aortic fitting 600 is placed in the wall of the aorta A. The aortic fitting 600 is generally tubular and has a first end 602 with an outer rim 604 and a strain relief zone 606 which allow connection to the aortic wall with surgical adhesive, sutures, or clamps similar to the manner described above for the connector 16. The second end 608 of the aortic fitting 600 has a series of barbs 610 or other mechanical fittings. A synthetic graft vessel G may simply be pushed over the barbs 610 to may a tight, leak-free connection. If desired, an external ring 612 may be placed down over the joint and sutured or otherwise connected to the graft vessel G and the aortic wall to provide strain relief.

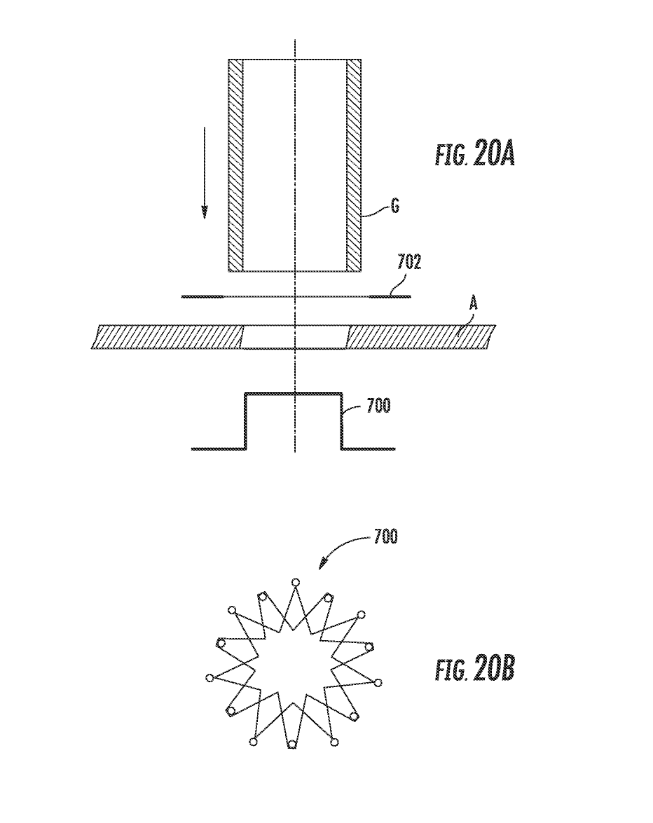

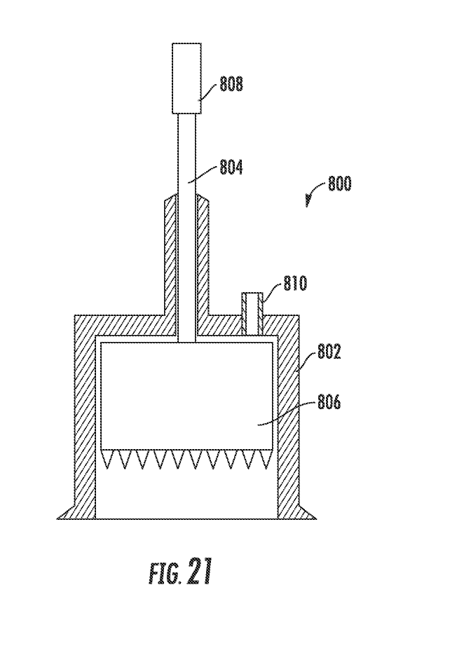

FIGS. 20A and 20B illustrate another method of making an aortic connection. An aortic fitting 700 is placed in the wall of the aorta A. The aortic fitting 700 is shaped like a short tee fitting and is made up of a framework of small struts, as seen in FIG. 20B. The aortic fitting 700 can be collapsed so that it can be inserted through the aortic wall and then will spring back to its original size. It is connected to the aortic wall with surgical adhesive, sutures, or clamps. The upstanding portion of the aortic fitting 700 fits inside a natural or synthetic graft vessel G and has several barbs, loops, or perforations or other mechanical fittings that allow connection of the graft vessel G thereto. If desired, an external ring 702 may be placed down over the joint and sutured or otherwise connected to the graft vessel G and the aortic wall to provide strain relief.

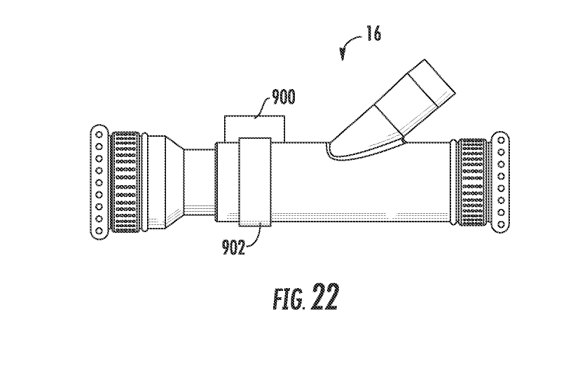

Regardless of what type of aortic connection is used, it is desirable to produce a uniformly round opening in the aorta A. This may be done with a cutter 800 depicted in FIG. 21. The cutter 800 has a housing 802 which is open at one end. It carries a shaft 804 that is free to rotate and translate up and down. A cylindrical blade 806, similar to a conventional “hole saw”, is mounted on the lower end of the shaft 804, and a handle 808 is provided at the upper end. A fitting 810 allows the connection of a suction source (not shown) to the interior of the housing 802. In operation, the cutter 800 would be placed against the aortic wall and suction applied to hold the housing 802 in place. The shaft 804 is then rotated while being fed downward. This results in a uniform, circular hole.

The CABG method and system described above does not require the use of harvested arteries or veins, and maintains the natural “hemodynamic” pulsatile flow of the blood with minimal reduction in the pulsations and blood flow velocity within the descending synthetic or engineered vascular tissue component.

Once the CABG is implanted as described above, it may be monitored with a variety of implantable sensors to determine if adequate flow is taking place it the graft vessels G and the connectors. For example, FIG. 22 shows a connector 16 with a transducer 900 clamped to its outer diameter with a band 902. Various known types of transducers can be used to monitor parameters such as blood flow velocity, temperature, oxygen level, and acoustics. One known type of sensor believed to be suitable for monitoring acoustics in the CABG is a digital hearing aid.

The information monitored from the transducers may be transferred externally by a wired or wireless connection. For example, an baseline derived flow rate or a baseline acoustic signature may be established. If the flow rate drops below the baseline amount, or substantial changes are observed in the acoustic signature, this would be a sign of blockage, leakage, or some other problem in the CABG.

The foregoing has described methods and apparatus for bypass grafts. While specific embodiments of the present invention have been described, it will be apparent to those skilled in the art that various modifications thereto can be made without departing from the spirit and scope of the invention as defined in the appended claims.

What is claimed is:

1. A vascular connector, comprising:
a main tube having a channel for fluid flow therethrough and opposed ends adapted to be connected to a vascular structure; and
at least one inlet tube having a channel for fluid flow therethrough, a proximal end intersecting the main tube, and a distal end adapted to be connected to a vascular structure, wherein the geometry of the intersection between the at least one inlet tube and the main tube is configured to enhance mixing of fluid flowing from the inlet tube with fluid flowing in the main tube.

2. The vascular connector of claim 1 wherein the main tube includes a throat of reduced cross-sectional area downstream of the proximal end of the inlet tube.

3. The vascular connector of claim 1 wherein an axis of the inlet tube is disposed at an acute angle to an axis of the main tube.

4. The vascular connector of claim 1 wherein the inlet tube is formed in a helical shape which surrounds the main tube.

5. The vascular connector of claim 1 wherein at least one of the main tube and inlet tube comprises first and second sections connected in a friction-fit telescoping relationship so as to be movable between collapsed and extended positions.

6. The vascular connector of claim 5 wherein:
the second section is received inside the first section;
the first section has a substantially constant inner diameter; and
the second section has a tapered outer diameter, such that the second section defines an annular sealing line contact with the first section.

7. The vascular connector of claim 6 wherein the first section includes an inwardly-extending retaining flange adapted to prevent withdrawal of the section second section therefrom.

8. The vascular connector of claim 5 wherein the first and second sections are free to rotate relative to each other.

9. The vascular connector of claim 1 wherein at least one end of the inlet tube or the main tube includes a protruding outer rim for engaging a vascular structure.

10. The vascular connector of claim 1 wherein at least one end of the inlet tube or the main tube includes a strain relief zone carrying a material adapted to promote cell growth therein.

11. The vascular connector of claim 10 wherein the strain relief zone carries collagen-hydroxyl-apatite tape thereon.

12. The vascular connector of claim 10 wherein the strain relief zone carries a fibrous scaffolding thereon.

13. The vascular connector of claim 1 wherein at least one end of the inlet tube or the main tube includes an open wire structure extending therefrom.

14. The vascular connector of claim 1 including at least one signal transducer attached thereto.

15. A method of manufacturing a vascular connector which includes a main tube having a channel for fluid flow therethrough and opposed ends adapted to be connected to a vascular structure, and at least one inlet tube having a channel for fluid flow therethrough, a proximal end intersecting the main tube, and a distal end adapted to be connected to a vascular structure, the method comprising:
providing a generally planar blank;
forming the blank into a shape comprising mirror-image half-sections of the main tube and inlet tube;
folding the blank along a centerline thereof to bring the half-sections together; and
bonding the free edges of the folded blank together.

16. The method of claim 15 further comprising applying a biocompatible coating to the blank before the step of forming.

17. The method of claim 15 wherein the free edges are bonded using electron-beam welding.

18. A coronary artery bypass graft, comprising:
a substantially rigid connector having:
a main tube with first and second ends; and
an inlet tube having a proximal end intersecting the main tube, and a distal end adapted to be connected to a vascular structure;
A synthetic vessel having a proximal end adapted to be connected to a first vascular structure, and at least one distal end connected to the distal end of the inlet tube.

19. The coronary artery bypass graft of claim 18 wherein the synthetic vessel includes a trunk at the proximal end and at least two branches each having a distal end connected to an inlet tube of a substantially rigid connector.
